(19) United States
(12) Patent Application Publication
(10) Pub. No.: US 2015/0173654 A1
(43) Pub. Date: Jun. 25, 2015

(54) ACTIVITY, POSTURE AND HEART MONITORING SYSTEM AND METHOD

(71) Applicant: SOLUTIONS NOVIKA, LA POCATIERE (CA)

(72) Inventors: RUDY BÉLANGER, SAINT-PACOME (CA); GERVAIS CONSTANT, LA POCATIERE (CA); GUILLAUME CARON, LEVIS (CA)

(21) Appl. No.: 14/576,338

(22) Filed: Dec. 19, 2014

Related U.S. Application Data
(60) Provisional application No. 61/918,742, filed on Dec. 20, 2013.

(51) Int. Cl. A61B 5/11; A61B 5/0402; A61B 5/00; A61B 5/0205

(52) U.S. Cl. CPC A61B 5/1117; A61B 5/0205; A61B 5/0402; A61B 5/1118; A61B 5/1121; A61B 5/6823; A61B 5/7246; A61B 5/0245

(57) ABSTRACT

A system and a method for monitoring activity and posture of a person in correlation with the person's heat activity, comprising a) simultaneously detecting first linear accelerations and first angular speeds of rotation of an upper part of the person's body, second linear accelerations and second angular speeds of rotation of a lower part of the person's body, and the person's electrocardiogram signal; and b) determining the person's activity and posture from the first and second linear accelerations, the first and second angular speeds of rotation, and at least one of: i) a derivation signal, ii) heart rate and iii) a respiratory rate, of the person from the electrocardiogram signal.

### CROSS REFERENCE TO RELATED APPLICATIONS

This application claims benefit of U.S. provisional application Ser. No. 61/918,742, filed on Dec. 20, 2013. All documents above are incorporated herein in their entirety by reference.

### FIELD OF THE INVENTION

The present invention relates to a system and a method for monitoring physical activity, posture and heart activity of a person.

### BACKGROUND OF THE INVENTION

Falls are a main health hazard for the elderly and are a major source of pains, functional problems and handicaps in the aging population.

Falls associating with aging are caused by a number of factors, including for example inadequate organization of the environment, i.e. typically homes, decreasing of the muscle mass, denutrition, vision problems, fear, and also a number of pathologies or physiological conditions that may be transient and related to rapidly fluctuating parameters or whose first symptoms appear in an insidious way. For example, secondary effects or drug interactions, irregular heart activity, Parkinson or Alzheimer diseases, and orthostatic hypotension may be involved. The absence of witnesses, a confused recollection of the event or a lack of cooperation for fear of hospitalization are other frequent elements that may make it difficult to understand causal factors of falls.

All fall events, beside their immediate criticality, contribute to increasing the risk of a loss of an elder's autonomy. An elder with a history of falls may also tend to lose confidence and be frightened and thus reduce her/his daily activities.

Moreover, detecting cardiac disorders such as arrhythmia, heart palpitation, tachycardia etc. . . . may be difficult, since symptoms most often occur when the person is in his/her living environment as opposed to when the person is in a hospital environment. While current holter cardiac devices are useful for recording such symptoms occurring outside of clinical grounds for a physicist to analyze, they do not give the physician information on the precise posture or activity the person was involved in at the time of the symptoms. The precise posture or activity the person was involved before and during the symptoms may significantly improve the diagnosis.

A method and a system for recording posture or activity of the person during and before occurrence of the symptoms may be very useful.

There is a need in the art for an activity and heart monitoring system and method.

The present description refers to a number of documents, the content of which is herein incorporated by reference in their entirety.

### SUMMARY OF THE INVENTION

More specifically, in accordance with the present invention, there is provided a method for monitoring activity and posture of a person in correlation with the person's heart activity, comprising a) simultaneously detecting first linear accelerations and first angular speeds of rotation of an upper part of the person's body, second linear accelerations and second angular speeds of rotation of a lower part of the person's body, and the person's electrocardiogram signal; and b) determining the person's activity and posture from the first and second linear accelerations, the first and second angular speeds of rotation, and at least one of: i) a derivation signal, ii) heart rate and iii) a respiratory rate, of the person from the electrocardiogram signal.

There is further provided a system for monitoring physical activity and posture of a person in correlation with the person's heart activity, comprising a first sensing unit configured to be positioned on an upper part of the person's body, a second sensing unit configured to be positioned on a lower part of the person's body, each one of the first and second sensing units comprising a 3-axes accelerometer and a 3-axes gyroscope, and an electrocardiogram sensing unit adapted to be positioned on the person's chest; a recorder connected to the first sensing unit, the second sensing unit and the electrocardiogram sensing unit; and an analysis program; wherein the first and second sensing units collect linear accelerations and angular speeds of rotation of the upper part and the lower part of the person's body respectively as the electrocardiogram sensing unit collects an electrical signal of the person's heart; the recorder receiving the linear accelerations, angular speeds of rotation data and the electrical signal of the person's heart, and the analysis program processing the linear accelerations and the angular speeds of rotation into data on the person's posture and activity; the analysis program processing the electrical signal of the person's heart into at least one of: i) a derivation signal, ii) heart rate and iii) respiratory rate, of the person, in correlation with the person's posture and activity.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 is a schematic view of a system according to an embodiment of an aspect of the present invention;

FIG. 2A is a schematic view of a recorder of a system according to an embodiment of an aspect of the present invention;

FIG. 2B is a schematic view of a recorder of a system according to another embodiment of an aspect of the present invention;

FIG. 3A is a diagrammatic view of a method of collecting data according to an embodiment of an aspect of the present invention;

FIG. 3B is a diagrammatic view of a method of processing collected data according to an embodiment of an aspect of the present invention;

FIG. 3C is a diagrammatic view of a method of collecting and processing data-according to another embodiment of an aspect of the present invention;

FIG. 3D is a diagrammatic view of a method of processing collected data according to another embodiment of an aspect of the present invention;

FIG. 4 is a diagrammatic view of a method according to an embodiment of an aspect of the present invention;

FIG. 5A shows an electrocardiogram (ECG) signal;

FIG. 5B shows cardiac frequency;

FIG. 5C shows activity level and respiratory rate;

FIG. 5D shows a top graph with lines for each body posture and a bottom graph with lines for each activity;

FIG. 6 shows an ECG signal;

FIG. 7A shows a first sequence of a reconstruction of a change in posture or of a fall;

FIG. 7B shows a second sequence in a reconstruction of a change in posture or of a fall;

FIG. 8A shows heart rate monitoring according to an embodiment of an aspect of the present invention;

FIG. 8B shows skewness coefficient determination according to an embodiment of an aspect of the present invention;

FIG. 9A shows atmospheric pressure as measured by a barometer of the system over time;

FIG. 9B shows derivation of the pressure measured as shown in FIG. 9A;

FIG. 9C shows change of height of the person's body corresponding to the pressure measured as shown in FIG. 9A and the derivation of the pressure as shown in FIG. 9B;

FIG. 10 illustrates the trunk verticality index;

FIG. 11A defines height parameters when the person is in a standing position;

FIG. 11B defines height parameters when the person is in a seated position;

FIG. 11C defines height parameters when the person is in a forward bent position;

FIG. 12 gives threshold values for first vertical displacements;

FIG. 13 gives threshold values for second vertical displacements; and

FIG. 14 lists downward transitions to be monitored for detecting fall events.

### DESCRIPTION OF ILLUSTRATIVE EMBODIMENTS

The present invention is illustrated in further details by the following non-limiting examples.

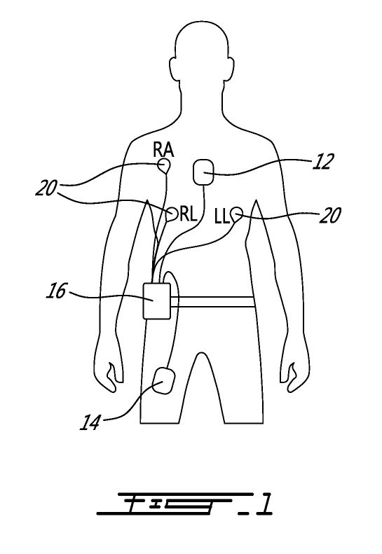

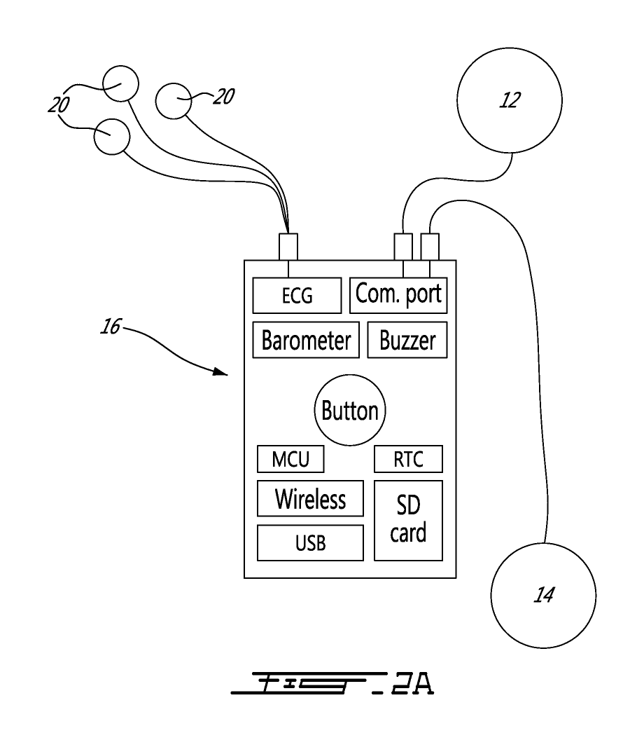

As shown in FIGS. 1 and 2, a system according to an embodiment of an aspect of the present invention comprises inertial sensing units 12 and 14, an ECG sensing unit 20, and a recorder 16.

In an embodiment illustrated in FIG. 1, a first inertial sensing unit 12 is adapted to be located on the upper part of a user's body, for example at the trunk of the user generally above the abdomen, while a second inertial sensing unit 14 is adapted to be located on the lower part of the user's body, below the waist generally at the thigh of the user.

The sensing units 12, 14 may be inertial measurement units (IMU) for example. They each comprise a combination of a 3-axis accelerometer and a 3-axis gyroscope. The 3-axis accelerometers measure linear accelerations along the three axes x, y, z of the body frame of the sensing units 12, 14 respectively, and the gyroscopes measure angular speed of rotation along the three axes x, y, z of the body frame of the sensing units 12, 14 respectively.

Using one units 12, 14 alone allows detecting that the user walks, i.e. a single unit 12 on the upper part of the user's body by detecting oscillations of the trunk of the user, and a single unit 14 on the lower part of the user's body by detecting angles of the thigh.

Combination of two sensing units 12, 14 at their respective location relative to the user's body as described hereinabove allows differentiating user's positions between seated, standing, and horizontal positions.

One of the first and second sensing units 12, 14, for example the sensing unit 12 located on the upper part of the user's body since the upper part of the user's body generally provides for a more stable locating position, may also comprise a magnetometer. By measuring intensity of the magnetic field along the three axes x, y, z of the body frame of the sensing units 12, 14 respectively, the magnetometer allows tracking the direction (east, west, north south) of the user's displacements, thus allowing assessing the trajectory of the user as he/she moves along, i.e. a path from a point A to a point B, of the user's displacements.

The ECG sensing unit 20 comprises electrocardiogram (ECG) electrodes 20 positioned on the user's chest, attached to the surface of the skin, which detect electrical activity of the heart, i.e. tiny rises and falls in the voltage between two electrodes placed either side of the heart positions shown in FIG. 1, referred to as right arm/left leg (RA/LL), allows obtaining a heart rate signal and the derivation DII. Other derivation may be selected. A third electrode (RL) is positioned to stabilize the signal, by common mode rejection of the differential amplification of the RA and LL voltage.

The recorder 16 may be worn at the belt by itself, i.e. separate from the sensor units 12, 14, as illustrated in FIG. 1. Alternatively, the recorder may integrate one of the two sensor units 12, 14 and, in such case, it is worn either on the upper part of the user's body or on the lower part of the user's body depending on which sensor units 12, 14 it integrates. Separating the recorder 16 from the sensor units allows a compact and lighter recorder, as well as compact and lighter sensing units.

As illustrated in FIG. 2, the sensing units 12, 14 are connected to the recorder 16 by communication ports, such as I2C connectors for example, and the ECG sensing unit 20 is connected to an ECG signal conditioning module (filtering and amplification) of the recorder 16 (FIG. 2a). Connection between the sensing units 12, 14 and the recorder 16 may also be wireless (see FIG. 2b).

The recorder 16 is generally a plastic casing with a battery carrier. It comprises a wire- or wireless communication interface, such as I2C or Bluetooth respectively for example, for connection with the first and second sensor units (see FIGS. 2a, b), storage means such as a SD card, a microcontroller with a real-time clock, a user interface comprising buttons (for activation/reset and wireless synchronization, typically at the back of the casing, and a front button for control request by the user, for example) and leds (for activation, wireless synchronization and battery level, for example). The front button for control request by the user is intended for use by the user in case of an event occurring to him/her, i.e. the user falls, in case of a malaise, loss of equilibrium or pain for example. A buzzer may be provided for confirming actuation of the front button.

The recorder 16 may further comprise a wireless communication protocol, such as Bluetooth or Wi-Fi for example, or a USB port for transmitting data received from the sensing units to a PC.

The recorder 16 may further comprise a barometer and a thermometer.

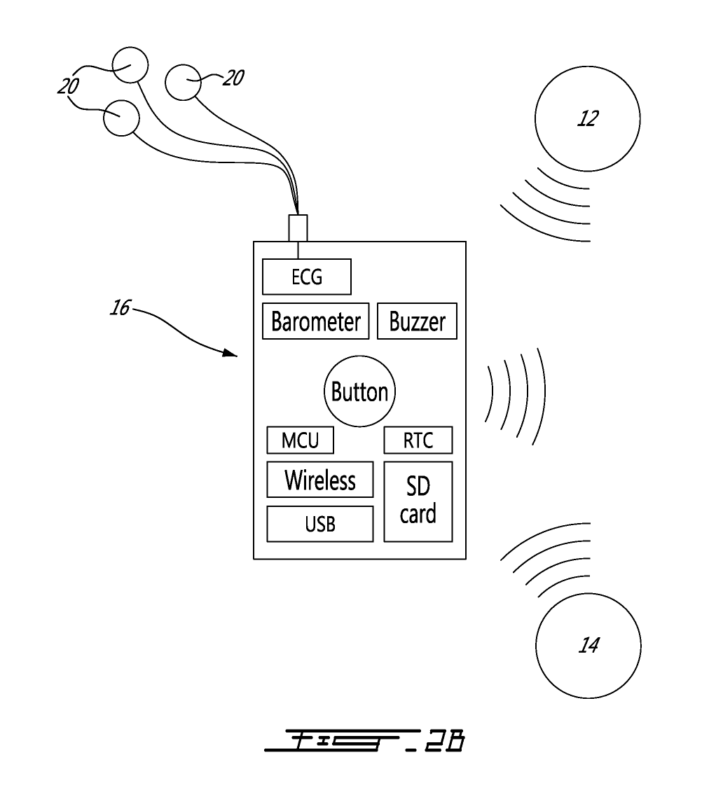

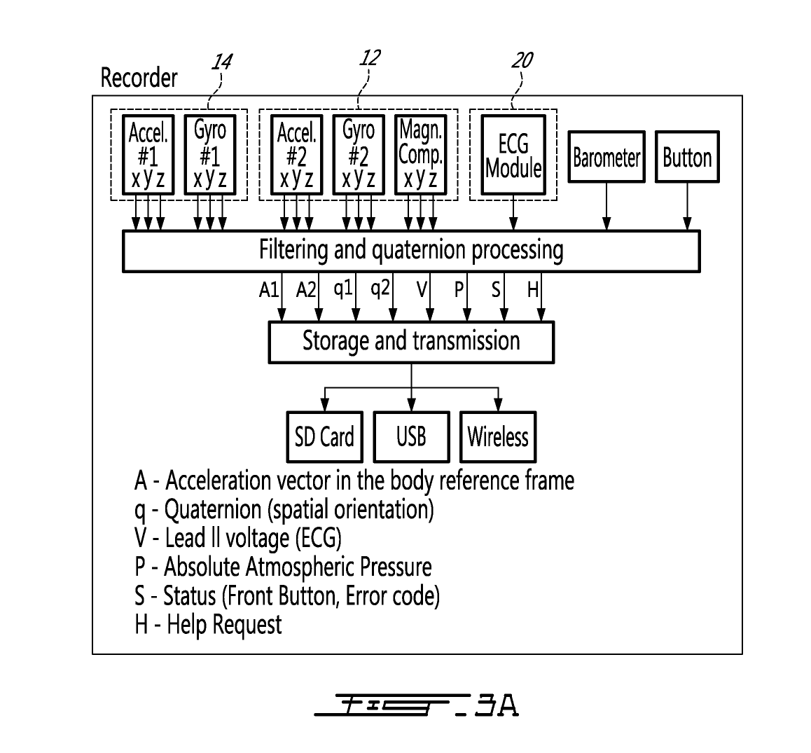

As illustrated in FIG. 3a, signals collected by the sensing units 12, 14 along the three axis x, y of the body frame of the sensing units 12, 14 respectively, z are filtered and processed to determine acceleration vectors A1 and A2 in the respective referential of the sensing units 12 and 14, quaternions q1 and q2 corresponding to spatial orientations of the sensing units 12 and 14 respectively. The electrocardiogram signal V collected by the ECG sensing unit 20 is processed to determine a DII derivation for example, of the ECG signal.

In case the recorder worn by the user comprises a barometer, data collected by the barometer are processed to determine variations in atmospheric pressure P, which may be used to assess altitude for tracking up and down movements of the user and fall events.

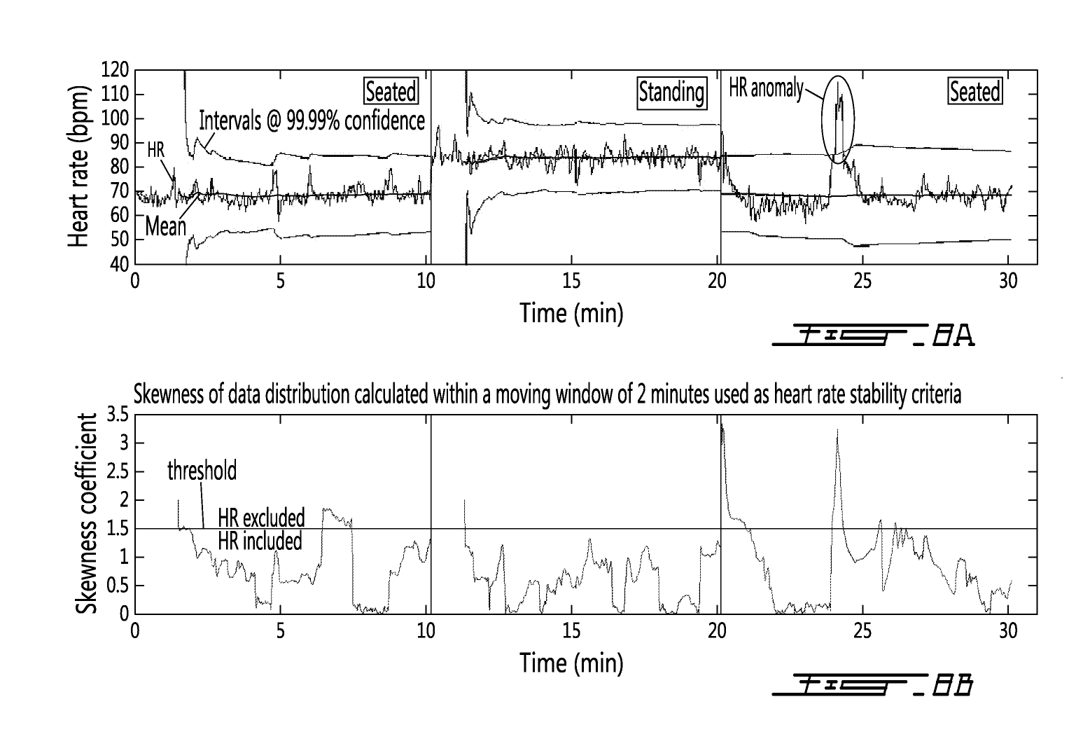

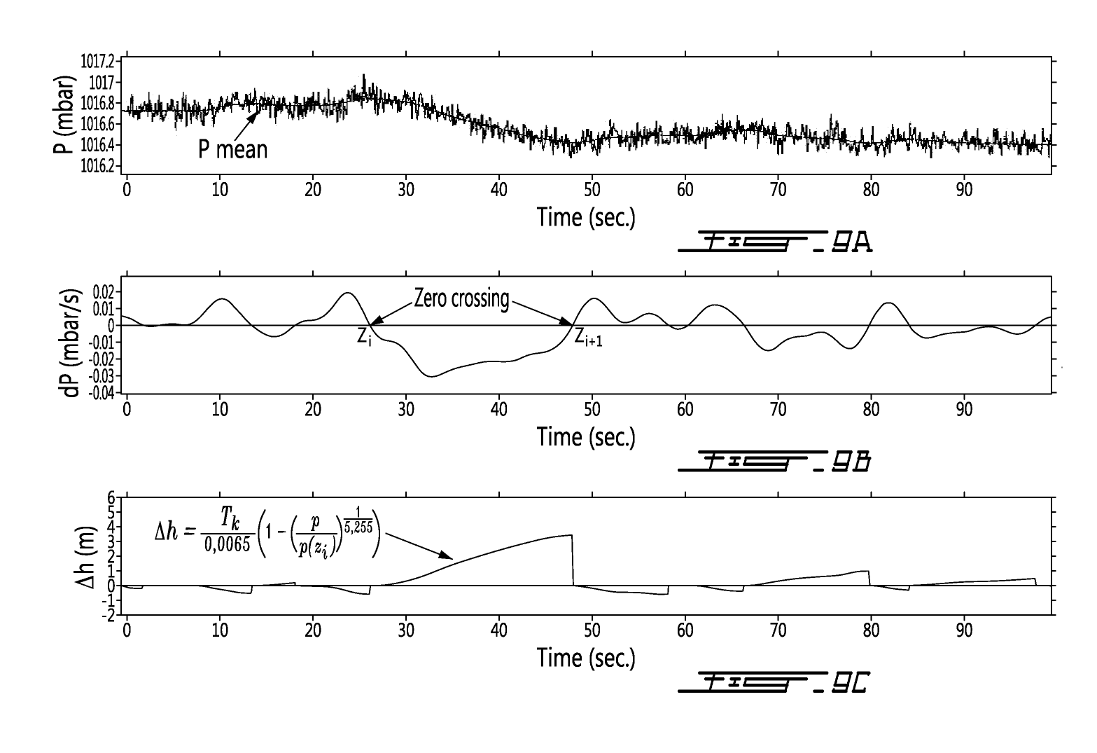

As illustrated in FIG. 9 in the case of a person moving from one floor to an upper floor for example, the barometer allows detecting the atmospheric pressure at the level of the person's body over time. In FIG. 9a for example, the pressure P is constant until the person goes up stairs at time about 30 s, which causes a drop of the pressure P, until the person reaches a final elevation at time about 48 s, where pressure P reaches a constant higher that before the person started going up (before time 30 s). From this measured change of pressures, each variation of the pressure, at time t=30 s and time t=48 s for example, corresponds to a null derivation dP(zi) as shown in FIG. 9b, which is stored in memory. The height variation Δh of the person's body can then be obtained using the following:

$$Δ h = \frac{{T}_{k}}{0.0065} ( 1 - {( \frac{p}{p ( {z}_{i} )} )}^{\frac{1}{5.255}} )$$

Where Tk is the temperature in Kelvin as measured by the thermometer and the constants are adapted to sea level conditions.

Signals from the front button (pushed button) are processed to signal an event experienced by the user, which gives rise to a status S and may result in a request for assistance H (local alarm, external alarm via smart phone, PC).

All data A1, A2, q1, q2, V, P, S, H thus obtained by the recorder from the collected signals can be stored in the storage means and/or transmitted. They may be accessible in real time with the wireless protocol or accessible using the USB port if any (see FIG. 3a) and then be processed by a PC program (see FIG. 3b).

Alternatively, as illustrated for example in FIG. 3c, the analysis program may be embedded in the recorder, which allows automatic generation of a help request, and also allows reducing the amount of data to be stored by the system.

The analysis program, either embedded in the recorder (FIG. 3c) or not (FIG. 3b) processes the acceleration vectors A1 and A2 in the respective referential of the sensing units 12 and 14, quaternions q1 and q2 corresponding to spatial orientations, and the voltage V of the DII derivation of the ECG, to determine the corresponding spatial position SP of the user, including the user's relative position, acceleration, velocity and displacement; the user's posture Pst, i.e. standing, seated, bent or lying down; the user's activity Act, i.e. at rest or still, walking, going up or going down; activity level AL between very low, low, moderate, high and very high; and characteristics of events FC such as falls (kind of falls, impact force, direction of the fall), in correlation with the heart activity as characterized by the derivation DII, the heart rate HR and the respiratory rate RR, and status data.

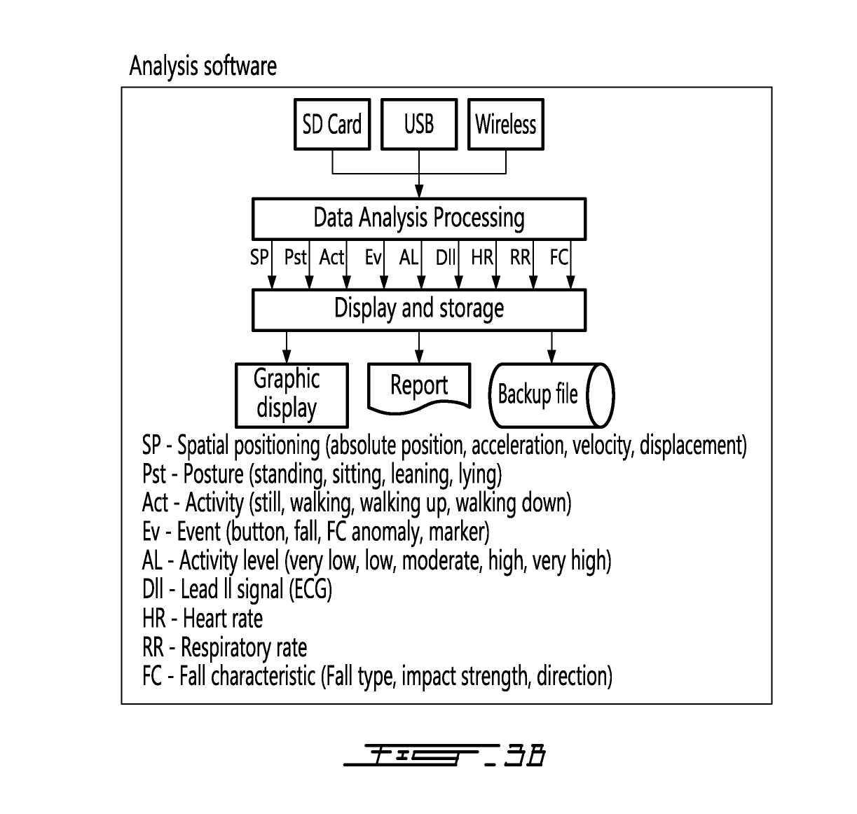

More precisely, as shown in FIG. 4, data collected by the sensing unit 12 located in the upper part of the user's body, for example on the user's trunk or chest, i.e. the acceleration vector A2 and the quaternion q2, are used to obtain the spatial positioning (SP) and a verticality index (angle ρ2) of the upper part of the user's body, i.e. displacement and orientation (in FIG. 4: Trunk INS stands for Inertial Navigation System for the trunk). The spatial positioning (SP) may include the relative position, i.e. a height from a ground reference or a distance from a reference, and a height of a vertical movement in case of a fall.

The signals from the sensing unit 14 located in the lower part of the user's body, for example on the user's thigh, i.e. the acceleration vector A1 and the quaternion q1, are used to obtain a verticality index (angle ρ1), i.e. orientation, of the thigh (in FIG. 4 Thigh AHRS stands for Attitude and Heading Reference System for the Thigh).

A verticality index ρ as shown in FIG. 10 is obtained from equation ρ=a cos(rₙ·r_{c}/|rₙ|·|r_{c}|) where vector rₙ is a vertical vector of the ground reference frame and r_{c} is a vector of the trunk toward the cranial axis obtained from the quaternion q₂ of the person's trunk (see FIG. 10). For example, in a Est, North, Up (ENU) axes convention, rₙ=[0,0,1] and the vector r_{c} is obtained from r_{c}=q₂·rₙ·q₂⁻¹.

The verticality indices ρ2 and ρ1 allow determining the posture Pst, i.e. the body stance of the user. For example, a seated position may be defined by ρ2<45°/ρ1>45°, a lying position by ρ2>45°/ρ1>45°, a bent position by ρ2>45°/ρ1<45°, and a standing position by ρ2<45°/ρ1<45°.

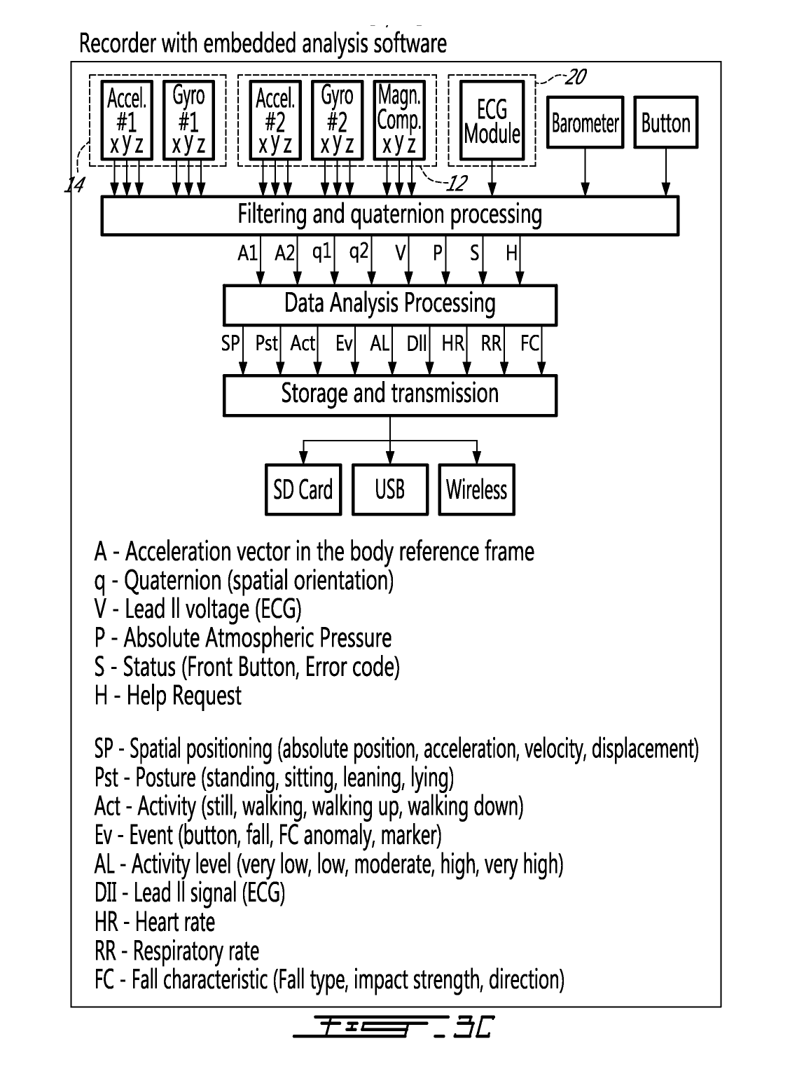

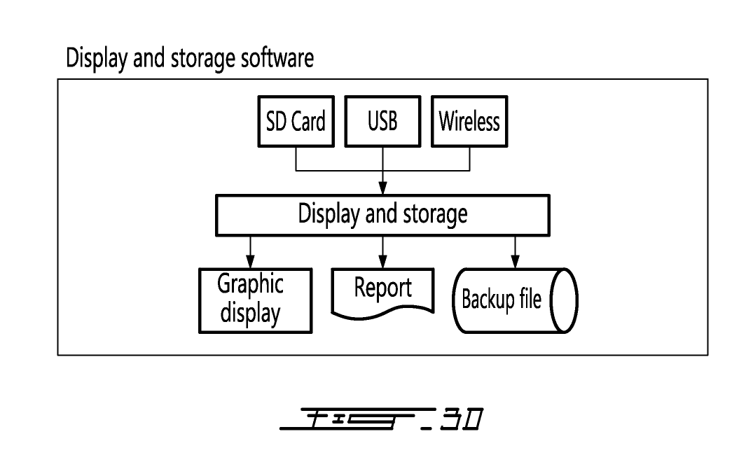

The quaternions q1 and q2 may further be used to determine further angles to further characterize the different postures (seated, lying bent and standing), to assess vertical pitch in standing position (forward or rearward bending) for example or horizontal roll in lying position (lying on a left or right side, or on the back or on the abdomen) example (FIG. 5d). In case of a fall, the quaternions q1 and q2 may also be used to determine if the fall occurred during a walking movement or during a transition between postures for example, and direction of the fall (backwards, forwards, sidewise etc.

With the body stance Pst, the acceleration vector A2 and the quaternion q2, the loss of height along the vertical direction, during a change in posture for example, may then be determined, by a double integration of the trunk acceleration (gravitational acceleration excluded) along the vertical axis of the ground reference frame, as determined from A2 and q2. The loss of height along the vertical direction is then compared to a predetermined range of normal loss of height. The range of normal loss of height is predetermined based on the user's characteristics or mensurations including the user's height when standing, the height of the sensing unit 12 on the user and the maximum angle of the user's upper body or trunk when the user is bent forward to his/her maximum ability in normal, predetermined, conditions.

By measuring the vertical displacement of the sensing unit 12 during changes of postures, normal displacements may be differentiated from displacements related to a fall. The vertical displacement of the sensing unit 12 during a change of posture depends on the person's body.

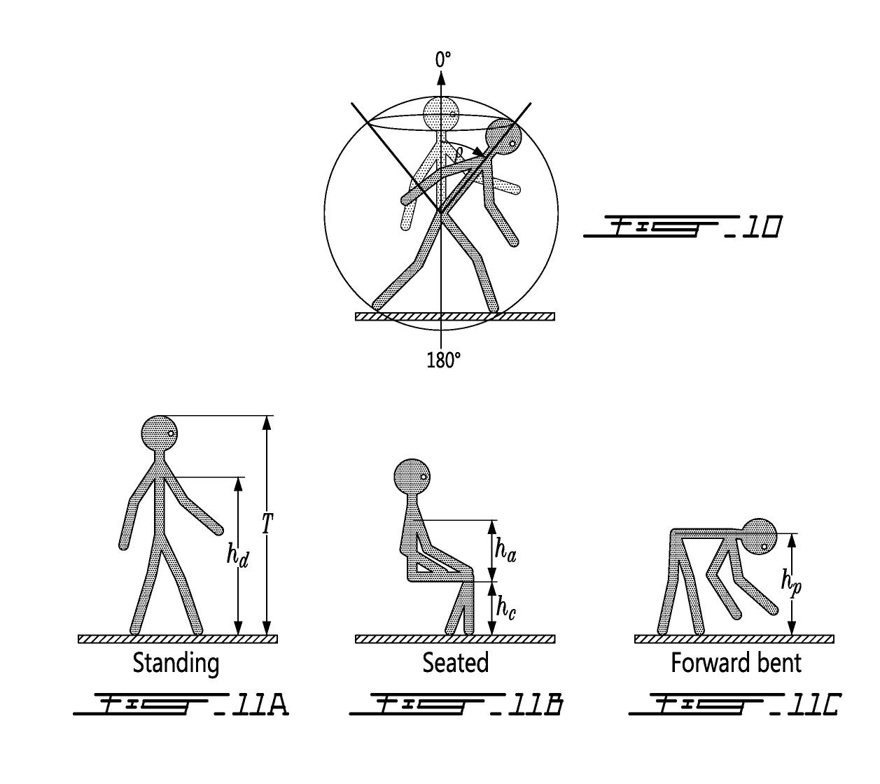

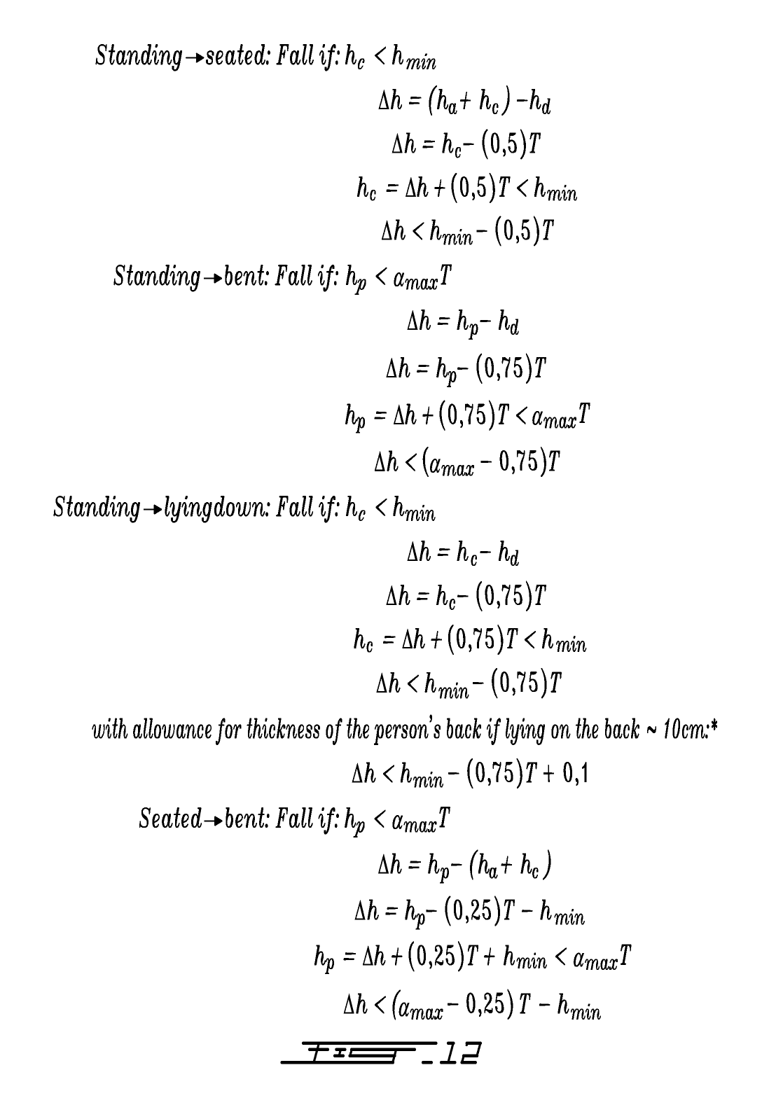

In FIG. 11, T is the height of the person, h_{d} is the height of the sensing unit 12 relative to the ground when the person is standing (FIG. 11a), hₐ is the height of the sensing unit 12 relative to person's buttock the when the person is seated and h_{c} is the height of the person's buttock relative to the ground the when the person is seated (FIG. 11 b), hₚ is the height of the sensing unit 12 relative to ground when the person is bent forward at 90° (FIG. 11c). h_{d}, hₐ and hₚ may be determined from the height T of the person using standard proportions for a human being for example, i.e. h_{d}=¾T, hₐ=¼ T and hₚ=½ T. From these, a normal vertical displacement Δh for a move from the standing position (FIG. 11a) to the seated position (FIG. 11b) of the person for example may be determined. In case of a downward displacement, the determination is done from the final height, i.e. as a negative displacement, as follows:

Δh=(hₐ+h_{c})−h_{d} i.e. Δh=h_{c}−1/2T

Table I below summarizes determination of normal vertical displacements Δh for different posture transitions:

| Transition | vertical displacements Δh |
|---|---|
| Standing to seated | (hₐ + h_{c}) − h_{d} |
| Standing to forward bent | hₚ − h_{d} |
| Standing to lying down | h_{c} − h_{d} |
| Seated to forward bent | hₚ − (hₐ + h_{c}) |
| Seated to lying down | −hₐ |
| Forward bent to lying down | h_{c} − hₚ |

A fall event is detected, with its characteristics (FC), when the loss of height along the vertical direction is larger than a threshold, i.e. normal loss of height, i.e. a loss of height corresponding to a transition from standing to seating for example and/or when a resulting height from the ground is detected to be below the typical height of a piece of furniture such as a bed or a chair, i.e. h_{c}=40 cm for example, and/or the bending angle is larger than the maximum angle of the user's trunk.

A minimal height hₘᵢₙ for the seated position (FIG. 11b) and a minimal height in the forward bent position, which depends on the maximum inclination angle ρₘₐₓ of the person's trunk (FIG. 11c) may thus be predetermined. In the case of a low chair or a bed in a lowest position, the minimal height hₘᵢₙ may be directly determined in relation to the low chair or the bed in the lowest position. Then, a fall is detected if, in a seated or lying position, h_{c}<hₘᵢₙ. In the case of the minimal height in the forward bent position, the height in the forward bent position hₚ is defined in relation to the inclination angle ρ of the person's trunk as follows:

hₚ=T−1/4T−1/4T(1−cos ρ)=T[3−(1−cos ρ)]/4

A height proportion factor α is thus be obtained for use in determining the height in the forward bent position as

α=[3−(1−cos ρ)]/4

hₚ is thus found to vary from (0.75)T in the standing position with ρ=0° to (0.25)T in a position with the head down with ρ=180°. hₚ is (0.50)T with ρ=90° (FIG. 11c). Then a fall is detected when, in a bent position:

hₚ<αₘₐₓT with αₘₐₓ=[3−(1−cos σₘₐₓ)]/4

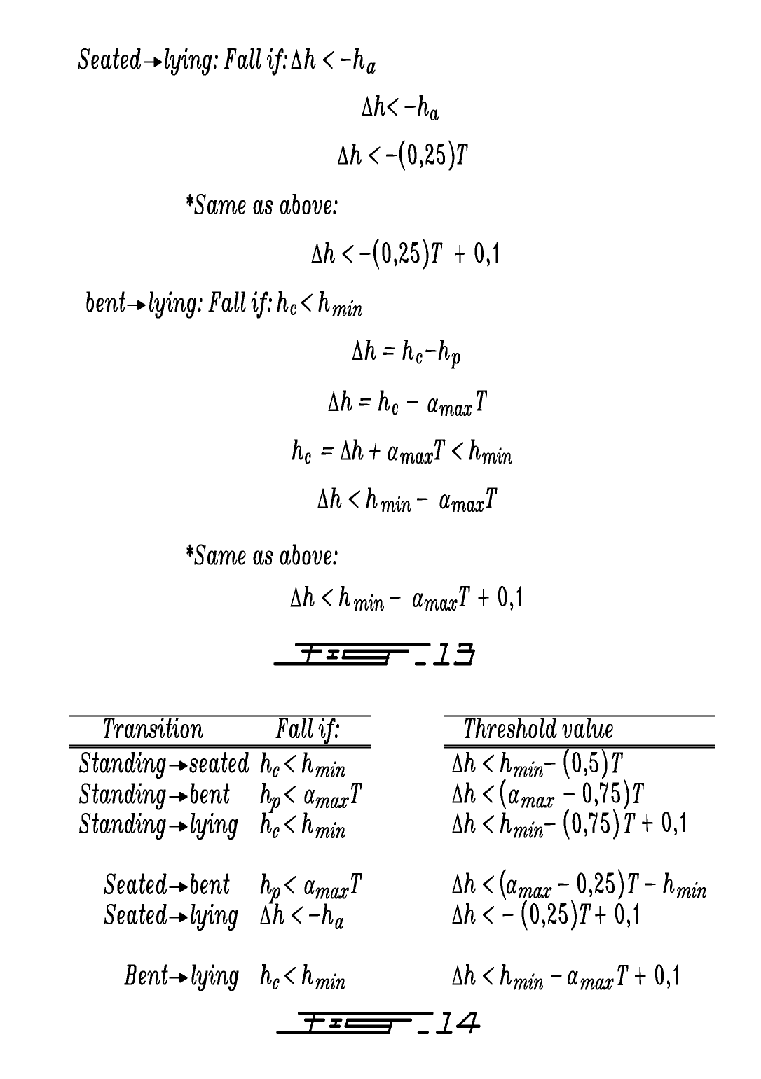

FIGS. 12 and 13 give the threshold values thus obtained for the vertical displacement Δh, and FIG. 14 lists transitions, i.e. displacement towards the ground, to be monitored.

Fall detection also comprises determining, from acceleration data measured by the accelerometers 12, 14, acceleration of the movement, force of the impact, maximum speed of the movement and duration of a transition between consecutive postures, for example between a standing position and a seated position, and detecting that at least one of these parameters is out of normal range, preset, in regards with the person. Fall detection also takes into account unusual postures, as defined by abnormal angles, for example in relation to a location in a building, i.e. lying down in a kitchen.

Fall detection may also use changes of pressure P as detected by a barometer integrated in the recorder for example. Correlated with data from the accelerometers 12, 14, data from the barometer may confirm occurrence of a fall event.

The verticality index ρ1 of the thigh, the acceleration vector A2 and the pressure P as measured by the barometer, which may be used to assess altitude for tracking up and down movements of the user, allow detecting activity of the user and determining its nature Act (i.e. stillness, walking, going up stairs, going down stairs), for example walking as evidenced by the verticality index ρ1 of the thigh, or going up as evidenced by a double integration of the acceleration vector A2 (gravitational acceleration excluded) in the ground reference frame. This double integration of the acceleration vector A2 between steps of the person as detected with the thigh pitch as determined from quaternion q1 allows characterizing displacement of the body along the vertical axis direction.

The level of the activity AL (very low, low, moderate, high, very high) can be determined using measurements of work or energy consumption for displacement of the trunk over a period of time, for example over a window of a to 10 minutes, as assessed from the accelerations and the weight of the trunk.

The signal V from the ECG electrodes is used to obtain the DII signal, for example, the heart rate (HR) and the respiratory rate (RR). The respiratory rate (RR) is obtained from the beat to beat variations in heart rate intervals which are primarily due to respiratory sinus arrhythmia (RSA) or from the ECG-Derived Respiration technique (EDR) as known in the art.

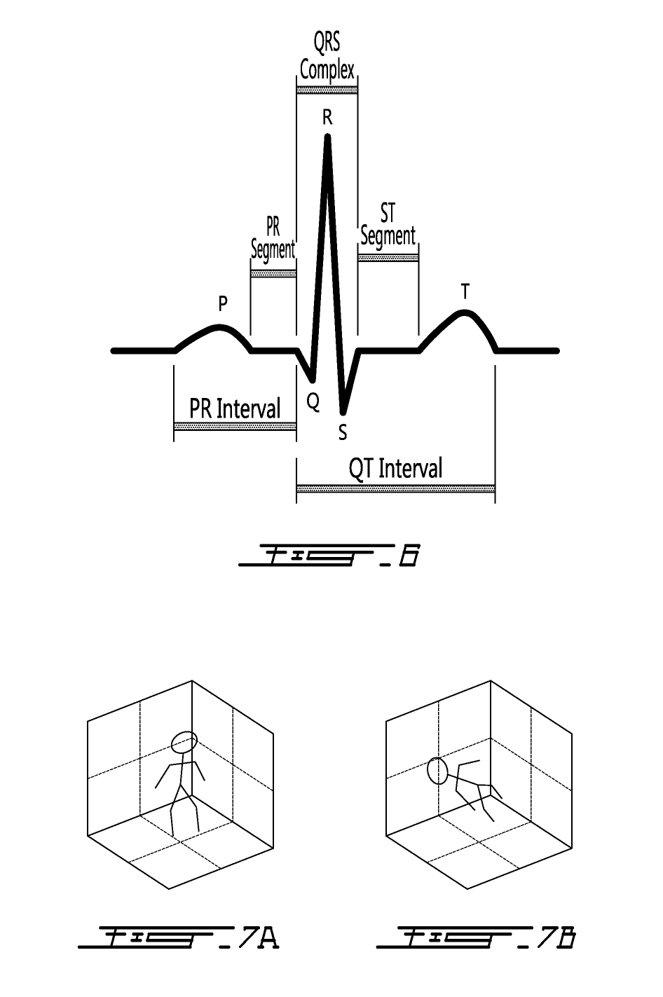

The heart rate (HR) is analyzed in correlation with the activity Act, activity level AL, and the posture Pst, to detect anomalies, based on statistical values available from general population to detect a potential problematic cardiac frequency, and based on the user's own data: by comparison with an average cardiac frequency range of the user in each posture, i.e. the signature of the user's average, over a 2 minutes window for example, heart activity in different spatial positions, postures and activities; the program marks any cardiac frequency outside an interval, defined for each position of the given user, as a ECG event as will be discussed hereon below in relation to FIG. 8. Then, the observer, for example a physician, may sort out the marked event by analyzing the different data, as explained hereinbelow in relation to FIG. 5 for example.

FIG. 8 show a simulation of a first transition from a seated position (first ten minutes) to a standing position (between minutes 10 and 20), and a second transition from the standing position to a seated position (minutes 20 to 30), of a person. In FIG. 8a, for each position, the mean heart rate of the person under test is shown, as well as an interval of normality. As can be seen, at the transitions at times 10 min and 20 min, the program redefines this mean rate and interval as characteristic features of the new posture. The seated position after time 20 min thus is defined with the same mean rate and interval as the first seated position before time 10 min. A heart rate anomaly (HR anomaly), simulated for the test by getting the tested person to maintain an effort for a few seconds before time 25 min, is shown as a peak.

In order to maintain the interval homogeneous in spite of such anomalies, for such anomalies the program uses an exclusion factor taking into account the skewness of the resulting measures, by using a moving time window, for example of 2 minutes as shown in FIG. 8b. In this window, if the data follow an asymmetric normal distribution, data above a skewness threshold in the determination of the mean heart rate and of the interval discussed hereinabove is excluded.

A fall detection combined with heart rate anomaly detection thus triggers detection of an event EV. The user pressing the push button (S) may also trigger detection of an event. An analyst may also manually trigger an event, using a manual marker in the analysis software.

ECG signal, cardiac frequency, respiratory rate and activity levels may be displayed as shown in FIGS. 5a, 5b and 5c respectively. The graph of activity levels and respiratory rate (FIG. 5c) is used to correlate variations in the cardiac frequency (FIG. 5b) that are a priori abnormal, with the postures (seated, standing, lying down) and the respiratory rate (i.e. breathing) of the user, which allows eliminating false ECG type events that may be caused by movement, effort or breathing artifacts. ECG details of a specific cardiac event marker may be observed on the ECG graph (FIG. 5a).

Data on activities and events may be presented under graphical form, showing the cardiac frequency (FC) and the time for each posture (standing, seated, laying down and bent) and activity (stillness, walking, going up stairs, going down stairs). In FIG. 5d, the top graph shows lines for each body posture, and the bottom graph shows lines for each activity. Events (automatically detected falls, detected abnormal cardiac frequencies, events marked by the user pressing the front button, or markers added by an analyst as mentioned hereinabove) are indicated by vertical lines (E1, E2, E3).

An observer, such a doctor for example, may thus cross-check the graphs of FIGS. 5b-5d to assess events that are automatically detected by the system.

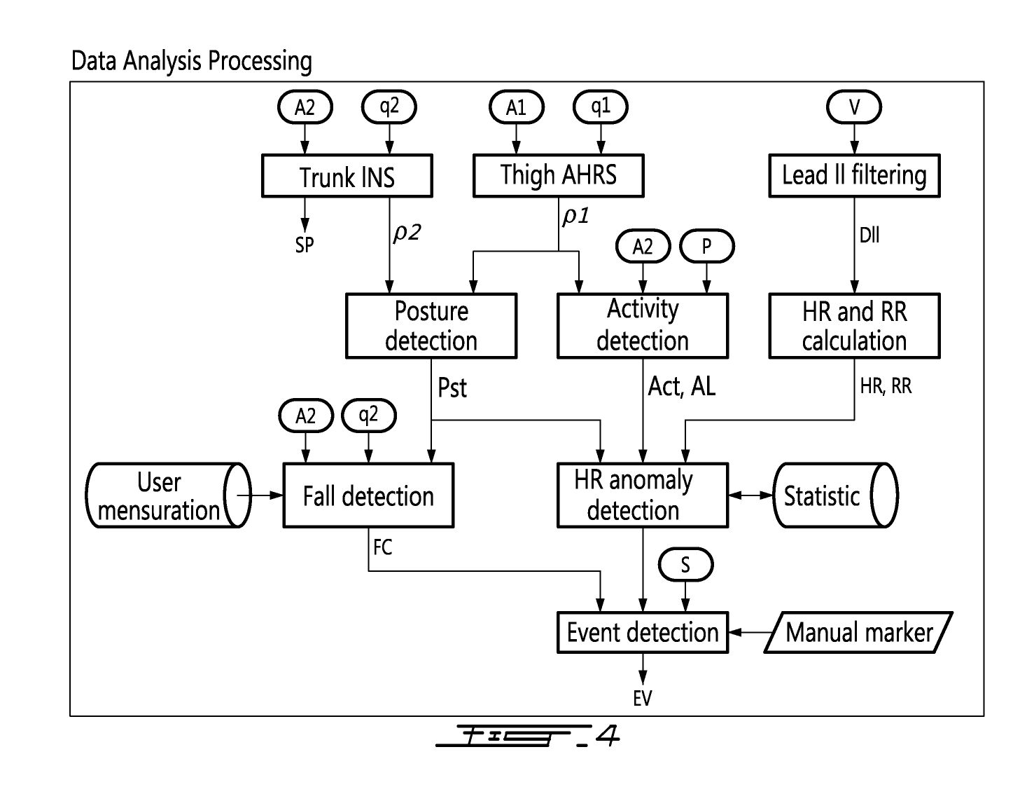

The signal from the ECG sensing unit 20 (see FIG. 5a) may also be directly available for a physician. The ECG signal as shown in FIG. 6 for example, may be used to detect a number of repetitive events or waves and delays between these waves: wave P, delay PR, complex QRS, inscription delay of the intrinsecoid deflexion, i.e. of the time between the QRS complex of the peak of wave R, QT delay, ST delay and T wave for example.

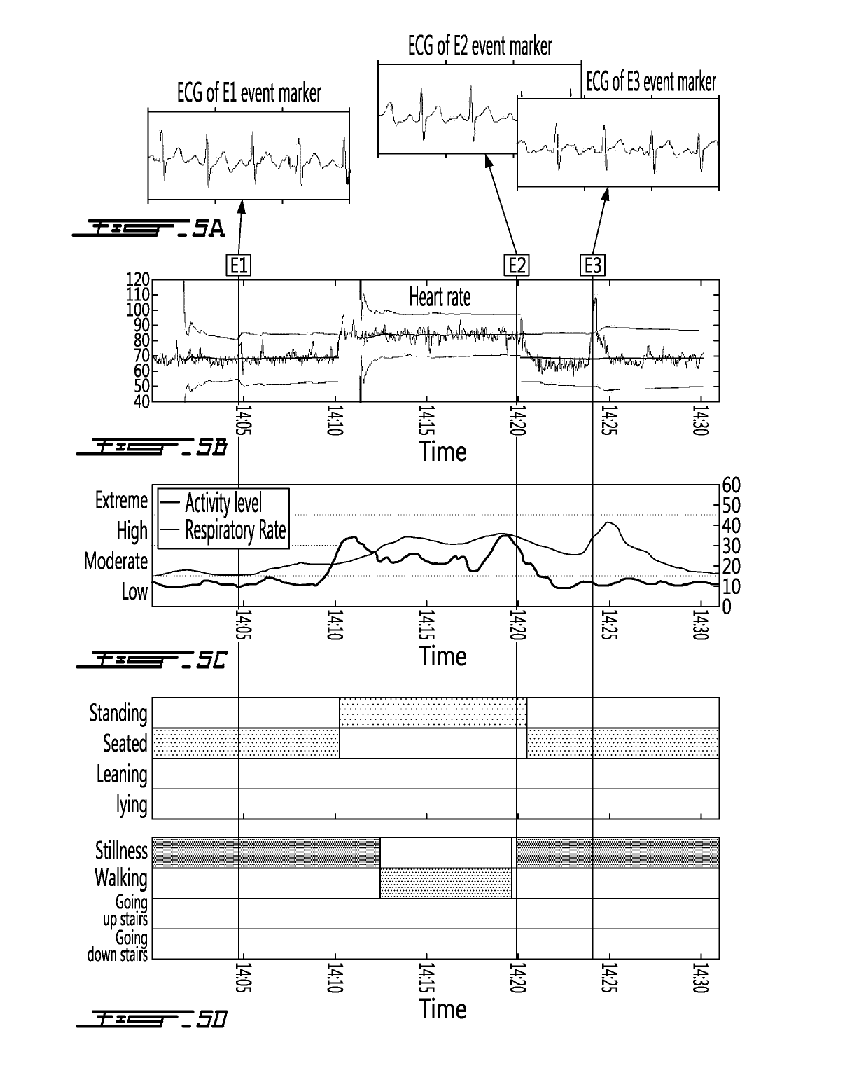

Reconstruction of changes in posture or of a fall may be done by displaying the event as recorded by the sensing units in real time. As shown in FIG. 7, data from the first sensing unit 12 and the second sending unit 14, i.e. the quaternions, are illustrated by orientations (see FIGS. 7a and 7b) of the top part, i.e. head shown as a circle, trunk shown as a straight line and arms shown as a three segment lines, and the bottom part, i.e. legs shown by a four segment lines, respectively, of a stick figure, for example, representing the user. Other user's representation may be used. Using a cursor on FIG. 5 for example, it is thus possible to go back or go forward of a sequence as shown in FIG. 7 so as to observe movements of the top part and of the bottom part of the user during an event. Thus spatial positions of the user may be correlated with the user's activity, the heart rate and respiratory rate to detect abnormal situations, for example using events that cross the different graphs of FIG. 5. Thus the present system provides a fall activity diagnosis tool, by allowing correlating physiological parameters, i.e. heart activity, to spatial positions, activities, and respiratory rate keeping track of a chain of such correlations leading to a fall and of the characteristics of such correlations at the moment of the fall.

The present system allows monitoring the heart activity of a user in relation to his/her activities including seated position, standing position and displacements, going up and down stairs, and lying position. It allows detecting falls and their pattern, rebuilding the fall and the events before the fall, recording pulse and P derivation of ECG, detecting pulse patterns, detecting patterns of events that may cause a fall.

The scope of the claims should not be limited by the embodiments set forth in the examples, but should be given the broadest interpretation consistent with the description as a whole.

1. A method for monitoring activity and posture of a person in correlation with the person's heart activity, comprising:
a) simultaneously detecting first linear accelerations and first angular speeds of rotation of an upper part of the person's body, second linear accelerations and second angular speeds of rotation of a lower part of the person's body, and the person's electrocardiogram signal; and
b) determining the person's activity and posture from the first and second linear accelerations, the first and second angular speeds of rotation; and at least one of: i) a derivation signal, ii) heart rate and iii) a respiratory rate of the person from the electrocardiogram signal.

2. The method of claim 1, wherein said step a) is performed using a combination of a first sensing unit configured to be located on the upper part of the person's body, a second sensing unit configured to be located on the lower part of the person's body, and an electrocardiogram sensing unit adapted to be located on the person's chest.

3. The method of claim 1, wherein said step b) comprises:
determining a first acceleration vector from the first linear accelerations, a second acceleration vector from the second linear accelerations, first and second quaternions from the first and second angular speeds of rotation respectively, and the derivation of the electrocardiogram signal; and
processing the first and second acceleration vectors, the first and second quaternions and the derivation of the electrocardiogram signal into corresponding spatial positions and activity of the person in correlation with the person's heart activity.

4. The method of claim 1, wherein said step b) comprises:
b1) determining first and second acceleration vectors from the first and second linear accelerations respectively, first and second quaternions from the first and second angular speeds of rotation respectively, and the voltage of the derivation of the electrocardiogram signal; and
b2) processing the first and second acceleration vectors, the first and second quaternions and the voltage of the derivation of the electrocardiogram signal into corresponding spatial positions and activity of the person in correlation with the person's heart activity;
wherein step b2) comprises:
determining a first verticality index of the lower part of the person's body using the first acceleration vector and the first quaternion;
determining a spatial positioning of the person and a second verticality index of the upper part of the person's body using the second acceleration vector and the second quaternion; and
determining the posture of the person from the second verticality index and the first verticality index.

5. The method of claim 1, wherein said step b) comprises:
b1) determining first and second acceleration vectors from the first and second linear accelerations respectively, first and second quaternions from the first and second angular speeds of rotation respectively, and the voltage of the derivation of the electrocardiogram signal; and
b2) processing the first and second acceleration vectors, the first and second quaternions and the voltage of the derivation of the electrocardiogram signal into corresponding spatial positions and activity of the person in correlation with the person's heart activity;
wherein step b2) comprises:
determining a first verticality index of the lower part of the person's body using the first acceleration vector and the first quaternion;
determining a spatial positioning of the person and a second verticality index of the upper part of the person's body using the second acceleration vector and the second quaternion;
determining the posture of the person from the second verticality index and the first verticality index; and
further characterizing the posture of the person using the first and second quaternions.

6. The method of claim 1, wherein said step b) comprises:
b1) determining first and second acceleration vectors from the first and second linear accelerations respectively, first and second quaternions from the first and second angular speeds of rotation respectively, and the voltage of the derivation of the electrocardiogram signal; and
b2) processing the first and second acceleration vectors, the first and second quaternions and the voltage of the derivation of the electrocardiogram signal into corresponding spatial positions and activity of the person in correlation with the person's heart activity;
wherein step b2) comprises:
determining a first verticality index of the lower part of the person's body using the first acceleration vector and the first quaternion;
determining a spatial positioning of the person and a second verticality index of the upper part of the person's body using the second acceleration vector and the second quaternion;
determining the posture of the person from the second verticality index and the first verticality index;
using the posture, the second acceleration vector and the second quaternion to determine a loss of height of the person's along a vertical direction;
comparing the loss of height with a predetermined loss of height; and
detecting a fall of the person when the loss of height along the vertical direction is larger than the predetermined loss of height.

7. The method of claim 1, wherein said step b) comprises:
b1) determining first and second acceleration vectors from the first and second linear accelerations respectively, first and second quaternions from the first and second angular speeds of rotation respectively, and the voltage of the derivation of the electrocardiogram signal; and
b2) processing the first and second acceleration vectors, the first and second quaternions and the voltage of a derivation of the electrocardiogram signal into corresponding spatial positions and activity of the person in correlation with the person's heart activity;
wherein step b2) comprises:
determining a first verticality index of the lower part of the person's body using the first acceleration vector and the first quaternion;
determining a spatial positioning and a second verticality index of the upper part of the person's body using the second acceleration vector and the second quaternion;
determining the posture of the person from the second verticality index of the upper part of the person's body and from the first verticality index;
using the posture, the second acceleration vector and the second quaternion, determining a loss of height of the person's body along a vertical direction,
comparing the loss of height with a predetermined loss of height;
determining, from the first and second acceleration vectors, acceleration of movement, force of impact, maximum speed of movement and duration of a transition between consecutive postures; and
detecting a fall when i) the loss of height along the vertical direction is larger than the predetermined loss of height and ii) at least one of the acceleration of movement, the force of impact, the maximum speed of movement and the duration of the transition is outside of a preset range.

8. The method of claim 1, wherein:
said step a) further comprises simultaneously detecting changes of pressure using a barometer adapted to be secured on the person's body; and
said step b) comprises:
b1) determining first and second acceleration vectors from the first and second linear accelerations respectively, first and second quaternions from the first and second angular speeds of rotation respectively, and the voltage of the derivation of the electrocardiogram signal; and
b2) processing the first and second acceleration vectors, the first and second quaternions and the voltage of a derivation of the electrocardiogram signal into corresponding spatial positions and activity of the person in correlation with the person's heart activity;
wherein step b2) comprises:
determining a first verticality index of the lower part of the person's body using the first acceleration vector and the first quaternion;
determining a spatial positioning and a second verticality index of the upper part of the person's body using the second acceleration vector and the second quaternion;
determining the posture of the person from the second verticality index and the first verticality index;
using the posture, the second acceleration vector and the second quaternion, determining a loss of height of the person's body along a vertical direction;
comparing the loss of height with a predetermined loss of height;
determining, from the first and second acceleration vectors, acceleration of movement, force of impact, maximum speed of movement and duration of a transition between consecutive postures;
detecting a fall when the loss of height along the vertical direction is larger than the predetermined loss of height and at least one of the acceleration of movement, the force of impact, the maximum speed of movement and the duration of the transition is outside of a preset range; and
confirming occurrence of a fall using the changes of pressure.

9. The method of claim 1, wherein:
said step a) further comprises simultaneously detecting changes of pressure as measured by a barometer adapted to be worn by the person; and
said step b) comprises:
b1) determining first and second acceleration vectors from the first and second linear accelerations respectively, first and second quaternions from the first and second angular speeds of rotation respectively, and the voltage of the derivation of the electrocardiogram signal; and
b2) processing the first and second acceleration vectors, the first and second quaternions and the voltage of the derivation of the electrocardiogram signal into corresponding spatial positions and activity of the person in correlation with the person's heart activity;
wherein step b2) comprises:
determining a first verticality index of the lower part of the person's body using the first acceleration vector and the first quaternion;
determining a spatial positioning and a second verticality index of the upper part of the person's body using the second acceleration vector and the second quaternion;
from the first verticality index, the second acceleration vector and the changes of pressure, detecting the nature of the person's activity and its level.

10. The method of claim 1, wherein detecting:
said step a) further comprises detecting pressure as measured by a barometer adapted to be worn by the person; and
said step b) comprises:
b1) determining first and second acceleration vectors from the first and second linear accelerations respectively, first and second quaternions from the first and second angular speeds of rotation respectively, and at least the voltage of the derivation of the electrocardiogram signal and the heart rate of the person from the electrocardiogram signal; and
b2) processing the first and second acceleration vectors, the first and second quaternions and the voltage of the derivation of the electrocardiogram signal into corresponding spatial positions and activity of the person in correlation with the person's heart activity;
wherein step b2) comprises:
determining a first verticality index of the lower part of the person's body using the first acceleration vector and the first quaternion;
from the first verticality index, the second acceleration vector and the pressure, detecting the nature of the person's activity and its level;
determining a second verticality index of the upper part of the person's body using the second acceleration vector and the second quaternion
determining the posture of the person from the second verticality index and the first verticality index;
analyzing the heart rate in correlation with the person's activity, the level of the person's activity and the posture to detect anomalies based on a comparison with an average cardiac frequency range of the person in each posture; and
detecting cardiac frequencies outside of the average cardiac frequency range of the user in each posture.

11. The method of claim 1, wherein:
said step a) further comprises detecting pressure changes as measured by a barometer adapted to be worn by the person; and
said step b) comprises:
b1) determining first and second acceleration vectors from the first and second linear accelerations respectively, first and second quaternions from the first and second angular speeds of rotation respectively, and at least the voltage of the derivation of the electrocardiogram signal and the heart rate; and
b2) processing the first and second acceleration vectors, the first and second quaternions and the voltage of a derivation of the electrocardiogram signal into corresponding spatial positions and activity of the person in correlation with the person's heart activity;
wherein step b2) comprises:
determining a first verticality index of the lower part of the person's body;
from the first verticality index, the acceleration vector and the pressure changes, detecting the nature of the person's activity and its level;
determining a second verticality index of the upper part of the person's body using the second acceleration vector and the second quaternion determining the posture of the person from the second verticality index and the first verticality index;
for each posture and activity, comparing the heart rate with an average cardiac frequency range of the user in each posture and activity; and
detecting cardiac frequencies outside of the average cardiac frequency range of the user in each posture and activity.

12. The method of claim 1, wherein:
said step a) further comprises detecting pressure changes as measured by a barometer adapted to be worn by the person; and
said step b) comprises:
b1) determining first and second acceleration vectors from the first and second linear accelerations respectively, first and second quaternions from the first and second angular speeds of rotation respectively, the voltage of a derivation of the electrocardiogram signal, the heart rate and the respiratory rate of the person from the electrocardiogram signal; and
b2) processing the first and second acceleration vectors, the first and second quaternions and the voltage of the derivation of the electrocardiogram signal into corresponding spatial positions and activity of the person in correlation with the person's heart activity;
wherein step b2) comprises:
determining a first verticality index of the lower part of the person's body;
determining the spatial positioning of the person and a second verticality index of the upper part of the person's body using the second acceleration vector and the second quaternion;
determining the posture of the person from the second verticality index and the first verticality index;
from the first verticality index, the second acceleration vector and the pressure changes measured by the barometer, detecting the person's activity, its nature and its level;
correlating the electrocardiogram signal, the heart rate, the respiratory rate and the activity level of the person to eliminate false electrocardiogram event.

13. The method of claim 1, wherein:
said step a) further comprises detecting pressure changes as measured by a barometer adapted to be worn by the person; and
said step b) comprises:
b1) determining first and second acceleration vectors from the first and second linear accelerations respectively, first and second quaternions from the first and second angular speeds of rotation respectively, the voltage of the derivation of the electrocardiogram signal, the heart rate and the respiratory rate of the person from the electrocardiogram signal; and
b2) processing the first and second acceleration vectors, the first and second quaternions and the voltage of the derivation of the electrocardiogram signal into corresponding spatial positions and activity of the person in correlation with the person's heart activity;
wherein step b2) comprises:
determining a spatial positioning and a verticality index of the upper part of the person's body from the acceleration vector and the quaternion;
determining a first verticality index of the lower part of the person's body;
determining the spatial positioning of the person and a second verticality index of the upper part of the person's body using the second acceleration vector and the second quaternion;
from the first verticality index, the second acceleration vector and the pressure changes measured by the barometer, detecting the person's activity, its nature and its level;
correlating the electrocardiogram signal, the heart rate, the activity level, the respiratory rate, the posture and the activity of the person.

14. A system for monitoring physical activity and posture of a person in correlation with the person's heart activity, comprising:
a first sensing unit configured to be positioned on an upper part of the person's body, a second sensing unit configured to be positioned on a lower part of the person's body, each one of said first and second sensing units comprising a 3-axes accelerometer and a 3-axes gyroscope, and an electrocardiogram sensing unit adapted to be positioned on the person's chest;
a recorder connected to said first sensing unit, said second sensing unit and said electrocardiogram sensing unit; and
an analysis program;
wherein said first and second sensing units collect linear accelerations and angular speeds of rotation of the upper part and the lower part of the person's body respectively as said electrocardiogram sensing unit collects an electrical signal of the person's heart; said recorder receiving said linear accelerations, angular speeds of rotation and said electrical signal of the person's heart, and said analysis program processing said linear accelerations and said angular speeds of rotation into the person's posture and activity; said analysis program processing said electrical signal of the person's heart into at least one of: i) a derivation signal, ii) heart rate and iii) respiratory rate, of the person, in correlation with the person's posture and activity.

15. The system of claim 14, wherein said recorder, from said linear accelerations and said angular speeds of rotation, determines first and second acceleration vectors and first and second quaternions corresponding to spatial orientations of each one of the first and second sensing units;
said analysis program processes the first and second acceleration vectors and the first and second quaternions to determine a corresponding spatial position and activity of the person in correlation with the person's heart activity.
